(19) United States
(12) Patent Application Publication
(10) Pub. No.: US 2026/0139312 A1
(43) Pub. Date: May 21, 2026

(54) Biversals in DNA Manipulation and Sequencing

(71) Applicants: Steven Benner, Gainesville, FL (US); Bang Wang, Gainesville, FL (US); Hyo-Joong Kim, Gainesville, FL (US)

(72) Inventors: Steven Benner, Gainesville, FL (US); Bang Wang, Gainesville, FL (US); Hyo-Joong Kim, Gainesville, FL (US)

(21) Appl. No.: 19/392,565

(22) Filed: Nov. 18, 2025

Related U.S. Application Data
(60) Provisional application No. 63/722,152, filed on Nov. 19, 2024.

(51) Int. Cl. C12Q 1/6874; C12Q 1/6876

(52) U.S. Cl. CPC C12Q 1/6874; C12Q 1/6876

(57) ABSTRACT

This invention provides compounds and processes that allow enzymes to catalyze the copying of DNA and RNA molecules to make complements where individual nucleotides are replaced in a rule-based fashion by other nucleotides, using biversal nucleotides that pair with two different nucleotides.

### CROSS-REFERENCE TO RELATED APPLICATIONS

This application claims priority to U.S. provisional patent application 63/722,152, filed 19 Nov. 2024, entitled “Biversals in DNA Manipulation and Sequencing”.

### FEDERALLY SPONSORED RESEARCH AND DEVELOPMENT

This work was supported by the National Institute of General Medical Sciences of the National Institutes of Health under Award Number R01 GM141391. The government has certain rights in the invention.

### JOINT RESEARCH AGREEMENT

Not Applicable.

### INCORPORATION-BY-REFERENCE OF MATERIAL SUBMITTED

An XML file for a “Sequence Listing XML” submitted via the USPTO patent electronic filing with the name (“Nonprovisional63722152_SeqList”), the date of creation (17 Nov. 2025), and the file size (17,899 bytes).

### BACKGROUND OF THE INVENTION

The field of this invention is nucleic acid chemistry, which concerns DNA, RNA, and their analogs. More specifically, it relates to binding between nucleic acid analogs of the instant invention and DNA target oligonucleotide molecules that contain cytosine (C), thymine (T), adenine (A), and guanine (G) heterocycles, as well as heterocycle analogs of these that have alternative hydrogen bonding patterns. Further, this invention relates to nucleotide analogs that, when embedded in an oligonucleotide probe, form pairs with Watson-Crick geometry with two size complementary bases. This invention further relates to the enzymatic synthesis of complexes formed when oligonucleotides containing these analogs bind to such targets.

### BRIEF SUMMARY OF THE INVENTION

By rearranging hydrogen bond donor and acceptor groups within a standard Watson-Crick geometry, DNA can add eight independently replicable nucleotides forming four additional pairs that are not found in standard Terran DNA (FIG. 1A, FIG. 1B, FIG. 1C). For many applications, the orthogonal pairing of standard and non-standard pairs offers a key advantage. However, other applications require standard and non-standard nucleotides to communicate with each other. This is especially the case when seeking to recruit high-throughput instruments (e.g. Illumina®), developed to sequence standard 4-nucleotide DNA, to sequence DNA that includes added nucleotides. For this purpose, PCR workflows are sought that replace non-standard nucleotides in (for example) a 6-letter DNA sequence by defined mixtures of standard nucleotides, a process called “transliteration”. High throughput sequencing can then report the sequences of those mixtures to bioinformatics alignment tools, which then infer the original 6-nucleotide sequence by analysis of the mixtures.

Unfortunately, the intrinsic orthogonality of standard and non-standard nucleotides often demand polymerases that violate pairing biophysics to do this replacement, leading to inefficiencies in this “transliteration” process. Thus, laboratory in vitro evolution (LIVE) using “anthropogenic evolvable genetic information systems” (AEGIS), an important “consumer” of new sequencing tools, has been slow to be democratized; robust sequencing is needed to identify the AegisBodies and AegisZymes that AEGIS-LIVE delivers.

This invention introduces a new way to connect synthetic and standard molecular biology: Biversal nucleotides. In one embodiment, a pyrimidine analog (pyridine-2-one, y) pairs with Watson-Crick geometry to both a non-standard base (2-amino-8-imidazo-[1,2a]-1,3,5-triazin-[8H]-4-one, P, the Watson-Crick partner of 6-amino-5-nitro-[1H]-pyridin-2-one, Z), and also pairs with a standard base that completes the Watson-Crick hydrogen bond pattern (2-amino-2′-deoxyadenosine, amA). PCR amplification of GACTZP DNA with dyTP delivers products where Z:P pairs are cleanly transliterated to A:T pairs. In parallel, PCR of the same GACTZP sample at higher pH delivers products where Z:P pairs are cleanly transliterated to C:G pairs. By allowing robust sequencing of 6-letter GACTZP DNA, this workflow will help democratize AEGIS-LIVE. Further, other implementations of the biversal concept can enable communication across and between standard DNA and synthetic DNA more generally.

### BRIEF DESCRIPTION OF DRAWINGS

FIG. 1A. By exploiting all hydrogen bonding patterns within the Watson-Crick geometry in duplex DNA formed by two oligonucleotide strands binding in an antiparallel orientation to form a duplex, or “double helix”, where each of the heterocycles in one strand interacts, generally by hydrogen bonding, with a heterocycle at the matched position on the other strand. The total number of DNA letters can been expanded from the standard four to 12 in an “anthropogenic evolvable genetic information system (AEGIS)”. These hydrogen bonding patterns are designated by the letters C, G, T, A, K, X, S, B, Z, P, V, and J. Shown here are the T:A and C:G pairs between standard heterocycles in the standard nucleotide (note that in RNA, T is replaced by U).

FIG. 1B. Shown here are the K:X and V:J pairs between the non-standard heterocycles K, X, S, V, and J.

FIG. 1C. Shown here are the S:B and Z:P pairs between the non-standard heterocycles S, B, Z, and P.

FIG. 1D. Chemical structures of standard and non-standard nucleotides which may interact with each other through proton gain and loss or by using biversal nucleotide analogs. (

AEGIS Z and standard G form a pair joined by three hydrogen bonds with Watson-Crick geometry if Z is deprotonated. However, AEGIS Z cannot pair with standard A, except by a wobble,

FIG. 1E. AEGIS P cannot interact with standard T or C by any geometry.

FIG. 1F. The biversal nucleotide y can form a pair joined by two hydrogen bonds with Watson-Crick geometry with both AEGIS P and amino-A, a functional analog of standard adenine. Thus, y acts as an intermediary to allow A to communicate with AEGIS P.

FIG. 2. New synthesis of dyTP. Reaction condition: (i) 2-(Phenylthio)-ethanol, Ph₃P, DEAD, THF, RT, 30 mins. 56%. (ii) (a) Pd(OAc)₂, AsPh₃, Ag₂CO₃, CHCl₃, 70° C., overnight; (b) Et₃N-3HF, THF, RT, 30 min; (c) NaBH(OAc)₃, CH₃CN/AcOH, RT, 30 min. 45% (total yield). (iii) H₂O₂, AcOH, 50° C., 3 hours. 80%. (iv) (a) DMTrCl, pyridine, DMF, RT, 1 h; (b) Ac₂O, pyridine, DMAP, DCM, RT, 30 mins; (c) DCA (3%), DCM, RT, 2 h. 68% (total yield). (v) (a) pyridine, CLOP, dioxane, RT, 15 mins; (b) pyrophosphate/tributylamine in DMF, RT. 20 mins; (c) iodine, water, pyridine, RT, 30 mins; (d) NH₄OH, RT, overnight. 10% (total yield).

FIG. 3A. Biversal nucleotide assisted sequencing of GACTZP DNA through transliteration. Z:P pairs were transliterated to C:G pairs via PCR amplification in the presence of five triphosphates d(A, T, C, G, Z)TP. This exploits a Watson-Crick pair between G and deprotonated Z. Transliteration of Z:P pairs to T:A pairs via PCR amplification with six triphosphates d(A, T, C, G, y, amA)TP.

FIG. 3B. PAGE-urea analysis of the PCR products from DNA templates (Nat or P-1) under various PCR conditions, followed by digestion with restriction endonuclease specific for the natural sequence after specific transliterations. Sanger sequencing is used to illustrate the sequencing of the P-1 template across a range of PCR conditions. Next Generation Sequencing (NGS) is used to measure the transliteration ratio of P to A or G under various PCR conditions.

FIG. 4. Chemical structures of biversal nucleotide analogs to communicate various standard or non-standard nucleotides. (A) Pyrimidines biversal nucleotides pair with purines nucleotides. (B) Purines biversal nucleotides pair with pyrimidines nucleotides.

### DETAILED DESCRIPTION OF THE INVENTION

Herein is describe the invention and its presently preferred embodiments. Further embodiments, and more examples of their use, are presented in Wang, B., Kim, H. J., Bradley, K. M., Chen, C., McLendon, C., Yang, Z., Benner, S. A. (2024). Joining natural and synthetic DNA using biversal nucleotides: Efficient sequencing of six-nucleotide DNA. J. Am. Chem. Soc. 146, 35129-35138. The content of this publication, including its supplementary material, is incorporated herein in its entirety by reference.

Molecules that bind other molecules are central to research, diagnostics, and therapeutics. Small molecule binders come via medicinal chemistry, which includes quantitative structure-activity analysis¹, distributed computing², and high throughput screening³. For protein binders, antibodies are the go-to molecules, especially if they are improved by laboratory directed evolution⁴. Other protein scaffolds may replace antibody protein scaffolds⁵.

Unfortunately, even today, medicinal chemistry remains a difficult way to get binders. Further, commercial antibodies, although powerful, often generate irreproducible outcomes that have been the subject of much discussion⁶. Peptide libraries are dominated by insoluble species⁷, as the amide backbone linkages with hydrophobic side chains create precipitates that form over folded states⁸.

Binding and catalytic molecules might be easier to develop if protein scaffolds were to be replaced by nucleic acid (NA) scaffolds. These, unlike protein scaffolds, are intrinsically soluble making them easier to evolve. Indeed, many have suggested that an early episode of life on Earth (the “RNA World”⁹) used RNA as the only encoded component of catalysts, and that these supported enabled a complex metabolism¹⁰. This model is consistent with the structures of RNA cofactors¹¹, the biosynthesis of proteins by catalytic ribosomal RNA¹², and examples of RNA catalysis in modern molecular biology¹³. With less confidence, some have suggested that Terran life itself emerged via the abiotic generation of an RNA molecule able to catalyze template-directed polymerization of RNA¹⁴.

This teases a question: If early life evolved binding and catalytic NAs to support complex lifestyles, could not a sophisticated molecular biology laboratory do the same, and more efficiently?Thus, Gold¹⁵, Szostak¹⁶, Joyce¹⁷, and others suggested that libraries of NA molecules might support laboratory in vitro evolution (LIVE), from which library components that bound a target receptor (aptamers) or catalyzed a target reaction (aptazymes) might be extracted and PCR amplified. Then, in a process analogous to lead development in medicinal chemistry or the maturation of primary antibodies, tighter binders and more efficient catalysts might be evolved by rounds of selection with mutation.

This approach had many successes¹⁸. However, over time, it became clear that natural NA is a poor substitute for proteins as a scaffold for evolving binders and catalysts. Natural NAs have just four building blocks, few functional groups, low information density, and poorly controlled folding. Indeed, in one case where an evolved DNAzyme was examined in detail, its limited catalytic power was found in large part to be due to folding ambiguity¹⁹. Separately, experiments that reduced the number of nucleotides to below 4 could produce RNA catalysts, but these were much less efficient²⁰. Conversely, adding functional groups to four nucleotide DNA makes them better²¹ catalysts.

Limited information density also confounded efforts to add to NAs functional groups thought to be important for binding and catalysis²². With standard 4-letter DNA, one of the four nucleotides participating in the two base pairs must carry that functional group. Since both pairs are needed to define a fold, functional groups ended up being present in greater numbers than needed, the “overdecoration problem”²³.

For example, Hirao found that a single hydrophobic nucleotide added to an aptamer could improve a typical affinity (50 nM) to sub-picomolar affinity²⁴. However, if a hydrophobic group is present on every thymine in an aptamer (for example, a SOMAmer²⁵), the resulting molecule can have limited binding specificity and lack the solubility that represents a strength of NAs as a scaffold for molecular evolution. Thus, SOMAmers are used in immobilized form on arrays, where the pattern of interaction with many low-specificity SOMAmers gives a high specificity read-out of analytes that might be present.

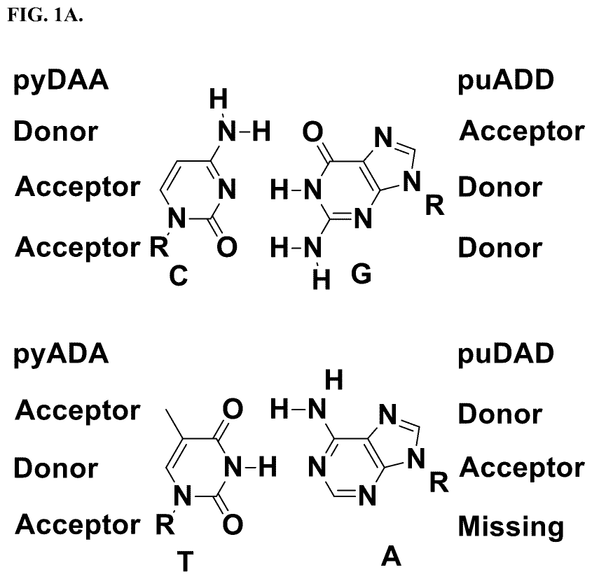

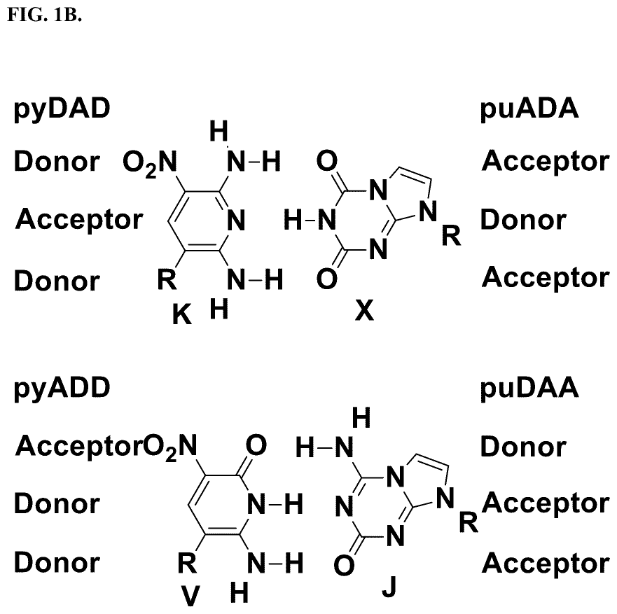

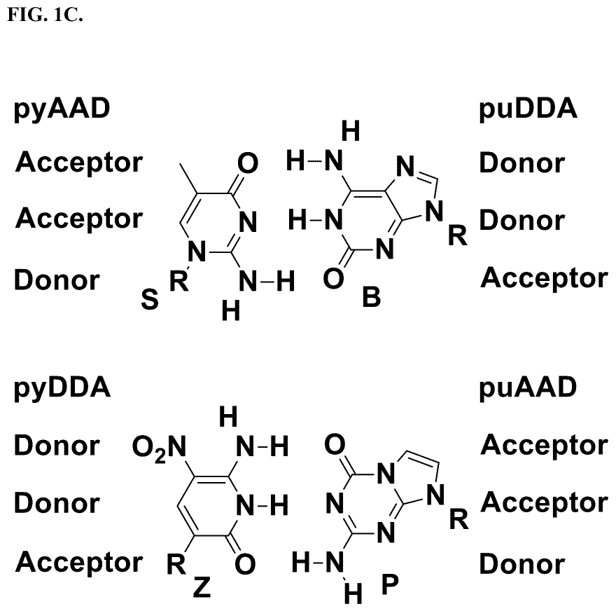

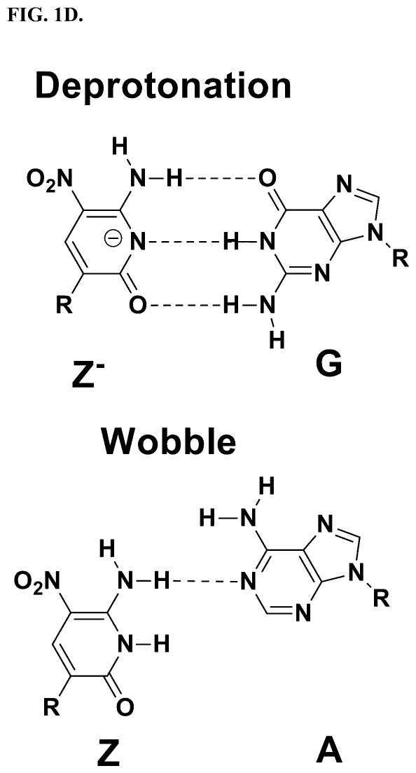

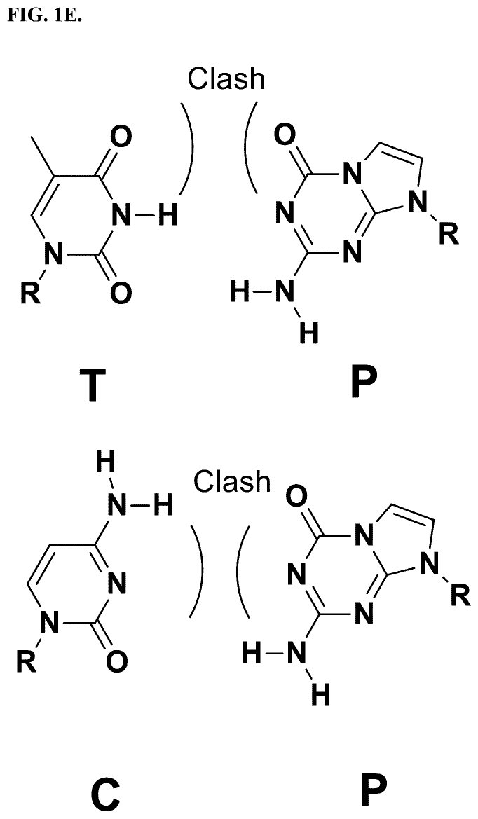

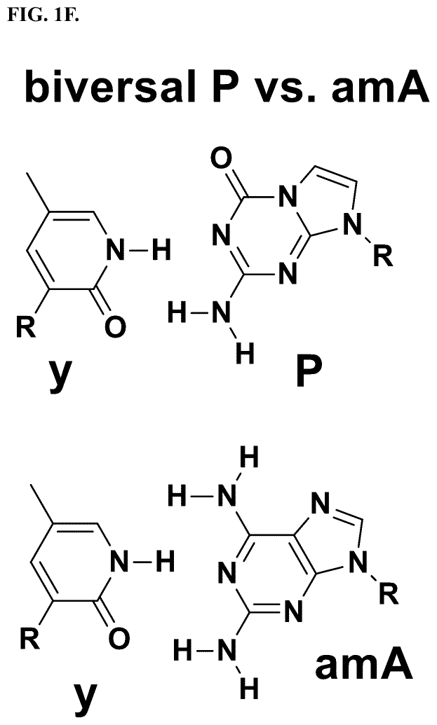

Anticipating these issues in 1987²⁶, the Benner lab proposed an “anthropogenic expanded genetic information system” (AEGIS)²⁷ that exploits all patterns of hydrogen bonding possible within a Watson-Crick pairing geometry; the four standard nucleotides in the two standard pairs exploit only two of these. Rearranging the hydrogen bond donor and acceptor groups adds eight independently replicable nucleotides forming another four independently replicable pairs to two pairs in standard Terran DNA (FIG. 1A). This completes the Watson-Crick base pairing concept²⁸ that Terran prebiotic chemistry had failed to complete²⁹. Functional groups could be added on a few nucleotides, with the others controlling folding, avoiding the over decoration problem. The repeating backbone charge would allow the system to evolve without encountering insolubility problems.

AEGIS improves nucleic acid scaffolds largely as anticipated. The higher information density of AEGIS DNA, its more rapid hybridization, and the orthogonality of added pairs separate from the standard pairs, have supported over $1 billion in diagnostics products³⁰. AEGIS-LIVE gives AEGISzymes³¹ and AEGISbodies³² that exploit higher information density to control folding³³. They also give new folds³⁴, including the recently discovered fZ-motif³⁵.

AEGIS libraries also proved to be richer sources of binding affinity and catalytic power. LIVE has been able to take increased advantage of this richness as our ability to PCR amplify AEGIS DNA with expanded alphabets without losing non-standard components have improved. Indeed, a recent study estimated that a GACTZP 6-letter AEGIS library is 100,000 times richer as a reservoir for ribonuclease-type catalysts than a standard 4-letter GACT library. In this case, Z in the AEGISzyme serves roles as a general acid-general base catalyst³¹. Re-inventing DNA required campaigns to synthesize AEGIS phosphoramidites and triphosphates to make AEGIS oligonucleotides³⁶ Campaigns of screening³⁷, protein engineering³⁸, and directed evolution³⁹ were required to get polymerases that replicate AEGIS DNA and RNA^{27,40}. Other efforts were required to develop the structural biology and solution biophysics of AEGIS pairs^{27,41}

Of course, a re-invented DNA also requires sequencing tools, preferably those that exploit “next generation sequencing” instruments that already benefit from enormous past investment to sequence four-letter DNA. In the ideal approach, an added pair Z:P pairs would, during PCR amplification, be transliterated in equal amounts to C:G and T:A pairs, respectively. The amplicon product would then be deep sequenced, and the sequences aligned by informatics to match those that arise from a single amplicon (but with Z:P transliterated to C:G) with those that arose from the same amplicon (but with Z:P transliterated to T:A). Sites containing C and T in the alignment would be called “Z” in the ancestral amplicon; sites containing G and A would be called “P” in that ancestor.

Robustly transliterating Z:P pairs to C:G pairs proved to be easy⁴². This transliteration exploited a pair having standard Watson-Crick geometry joined by three hydrogen bonds between G and deprotonated Z (FIG. 1D. top). This was possible because Z has a relatively low pKₐ (approximately 7.8)⁴².

Unfortunately, conditions to robustly transliterate Z:P pairs to T:A pairs were difficult to find. This transliteration would require either a P:T mismatch or a Z:A mismatch. A P:T mismatch requires a C═O×O═C clash (FIG. 1E. top). A Z:A mismatch requires a non-canonical “wobble” geometry joined by a single hydrogen bond (FIG. 1D, below). Thus, while forcing this translation to be done by depriving a PCR system of dZTP and dPTP was able to sequence AEGISzymes³¹ and AEGISbodies³² in expert laboratories, its workflow complexity is not adequate to democratize AEGIS-LIVE.

Therefore, an alternative sequencing approach was developed⁴³ to exploit enzymatic deamination of natural cytidine (C), transliterating it to uridine (U). As before, during PCR amplification, AEGIS Z is transliterated to cytosine (C), involving the robust mismatching of standard G with deprotonated Z. Then, bioinformatic alignment with the direct PCR products was used to call the bases in the parent sequences. Since all of the existing C's had been transliterated to “T”, the sequences could be deconvoluted by comparing the sequences with and without deamination.

Unfortunately, the deamination process gave amplicon products rich in A and T. This gave to poor quality high throughput sequencing reads.

These outcomes motivated the search for inventions to do robust transliteration and, in particular, transliterate Z:P pairs to T:A pairs. The idea proposed here involves “biversal nucleotides”. Biversal nucleotides are nucleobase analogs that form Watson-Crick pairs with two size complementary nucleobases without being deprotonated or protonated, without tautomerism, but rather only by strategic manipulation of hydrogen bonding groups⁴⁴.

Here, we exemplify this general idea by robustly solving the six-letter GACTZP sequencing problem. This required a biversal pyrimidine analog that could pair both with AEGIS P and standard A to allow robust transliteration of Z:P pairs to T:A pairs.

To construct this biversal analog, we began with a C-glycosidic analog of thymidine first synthesized by Solomon and Hopkins⁴⁵ and studied by Ishikawa⁴⁶ et al. This is a C3-pyridine-2-one C glycoside (y, FIG. 1F) that is missing a “top” exocyclic hydrogen bonding group. Thus, y cannot form the “top” hydrogen bond to the N⁶ amino group of adenine. Further, since natural adenosine is missing a minor groove “bottom” hydrogen bonding amino group, the y:A pair is joined by only one hydrogen bond. This is insufficient to compete with hydrogen bonds between the nucleobases and solvent water.

However, if adenine is replaced by aminoadenine, the y:amA pair is joined by two hydrogen bonds. These can compete with hydrogen bonding to water. Further, y forms a pair with Watson-Crick geometry with P, also joined by two hydrogen bonds (FIG. 1F). Thus, y is a “biversal” nucleobase. It can pair with two hydrogen bonds and a Watson-Crick geometry with two purine analogs. As one of these is standard (A) and the other is non-standard (P), y prospectively might allow the two DNAs to communicate with each other.

This led to experiments showing the utility of compositions of matter that are two oligonucleotides that are hybridized in a duplex, where (as is standard in DNA duplexes, or double helices in general, following the well-known Watson-Crick structure for the double helix the strands of said two oligonucleotides have an antiparallel orientation, and where each nucleotide in one strand forms a hydrogen bonded pair with an oligonucleotide in the other strand, where those pairs include y paired with diaminopurine, or y paired with 2-amino-imidazo-[1,2-a]-1,3,5-triazin-(8H)-4-one, the systematic name for the heterocycle represented in the drawings trivially as “P”.

Further, inventive processes were developed to create oligonucleotides with those pairs by the template-directed incorporation of y opposite P, or y opposite diaminopurine, or diaminopurine opposite y, or P opposite y, where the process of incorporation involves (respectively) the incubation of the triphosphate of y (yTP) opposite P in a DNA template, or yTP opposite diaminopurine in a DNA template, or the triphosphate of diaminopurine (amATP) opposite y in a DNA template, or the triphosphate of P (PTP) opposite y in a DNA template, where the process requires a DNA polymerase, the triphosphates of other nucleotides as required to allow the complementary strand synthesis to be completed by template-directed DNA synthesis.

Various of the preferred polymerases are detailed in the examples, and in Wang et al. (op cit). While exemplification is provided for deoxyribonucleotides, following the appropriate experimentation, these pairings are expected to also find utility when one or both of the complementary strands are RNA, of analogs of DNA with various alternative sugars. Likewise, tagged versions of P, y, and adenosine, the last in its 7-deaza form, may also participate in pairing having utility, as well as in processes that make these pairs by template-directed primer extension catalyzed by the appropriate DNA polymerase, RNA polymerase, or reverse transcriptase.

The utility of these processes is shown here by applying the processes and the oligonucleotide duplexes that are created by them to the task of sequencing GACTZP 6-letter DNA with controlled transliteration that connects AEGIS DNA with standard DNA. We transliterate Z:P pairs to T:A pairs during PCR amplification with addition of dyTP, damATP, and dPTP (optionally). In GACTZP DNA, template P first pairs with y in the absence of dZTP. In the second step, y (now in a template) pairs with amATP, directing its incorporation into a complementary strand. Then, amA, now in the template, pairs with standard dTTP, which can template the incorporation of standard dATP. These events, all in one pot, transliterate Z:P pairs to T:amA pairs, which are then sequenced as T:A pairs. The overall result is biversal nucleotide assisted sequencing (BNA-Seq) for GCATZP AEGIS DNA.

This work with biversals shows how inter-base hydrogen bonding can be manipulated to solve a specific problem with the first theme: letting existing high throughput sequencing platforms sequence GACTZP DNA. However, the concept can be generalized.

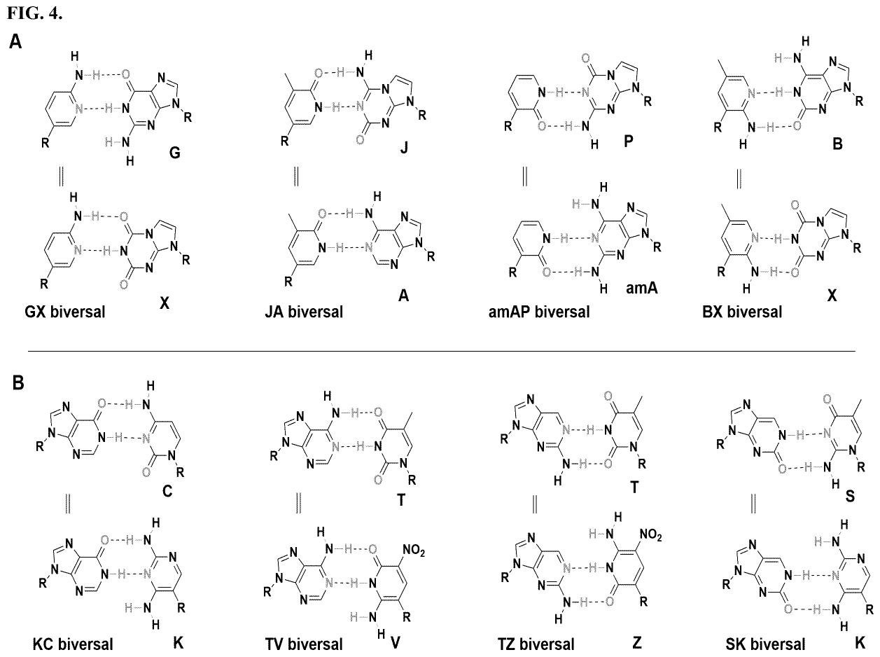

Central to the biversal concept is the observation that unless a nucleobase forms at least two hydrogen bonds to its partner, hydrogen bonding to solvent can compete effectively with inter-base hydrogen bonding⁴⁷. This constrains the design of biversals. With these constraints, FIG. 4 applies the same concept across the 12 letter DNA alphabet. Thus, careful manipulation of hydrogen bonding groups on pyrimidine analogs gives biversals that should allow G/X (pyDA-), J/A (pyAD-), P/A (py-DA) and B/X (py-AD) interconversion (D represent hydrogen bond donor group, A represent hydrogen bond acceptor groups). As no purine analog presents a DDD hydrogen bonding pattern or a AAA hydrogen bonding patterns, the py-DD and py-AA heterocycles are not biversal.

Likewise, careful manipulation of hydrogen bonding groups on purine analogs gives biversals that should allow C—K, S—K, T-Z, and T-V interconversion. Since there is no pyrimidine analog that presents a AAA hydrogen bonding pattern, the G analog with the top hydrogen bonding group deleted has no second partner.

Note, just as we can supplement biversality with bases that are deprotonated in the Z system, we can do this with other systems. For example, the diaminopyrimidine implementation of the K (DAD) hydrogen bonding pattern is a relatively strong base, becoming protonated with a pKa of ˜7. In its protonated form, this heterocycle presents a DDD hydrogen bonding pattern.

Entirely separately from this, reviewers have noted that a polyelectrolyte backbone, which the Polyelectrolyte Theory of the Gene²⁸ posits as necessary for any informational biopolymer to be able to support Darwinian evolution, seems to have difficulty forming core folds through backbone-backbone interactions. Of course, such core folds are exactly what produce compact structures in proteins, including beta sheets and alpha helices. It is commonly thought that such core folds are essential for very tight binding or truly effective catalysis.

Expanded genetic alphabets offer an opportunity to explore this hypothesis. With polyelectrolytes, core folds can arise from side chain-side chain interactions. With standard 4-nucleotide DNA, the G quadraplex⁴⁸ is the only one available near neutral pH.

However, AEGIS offers many more base-base interactions that give folds. These include assemblies formed by isoguanosine⁴⁹ (AEGIS B), fat and skinny pairs^{34b} (AEGIS T:K, S:Z, and V:C), and the fZ-motif³⁵. Further, AEGIS components that have hydrophobic tags can form hydrophobic cores by base tag-base-tag interactions if they are sparingly introduced so as to not cause overdecoration and precipitation that overwhelms the intrinsic solubility of the polyanionic backbone.

This invention introduces a new idea, “biversality”, for sequencing in expanded DNA particular, but also to connect the molecular biological universe of the natural world to the molecular biological universe being re-invented by synthetic biologists. The biversality concept can connect many partners within AEGIS DNA, as it does within natural DNA, and between AEGIS and natural DNA. Thus, it introduces a new concept into the design of nucleic acid systems.

### Examples

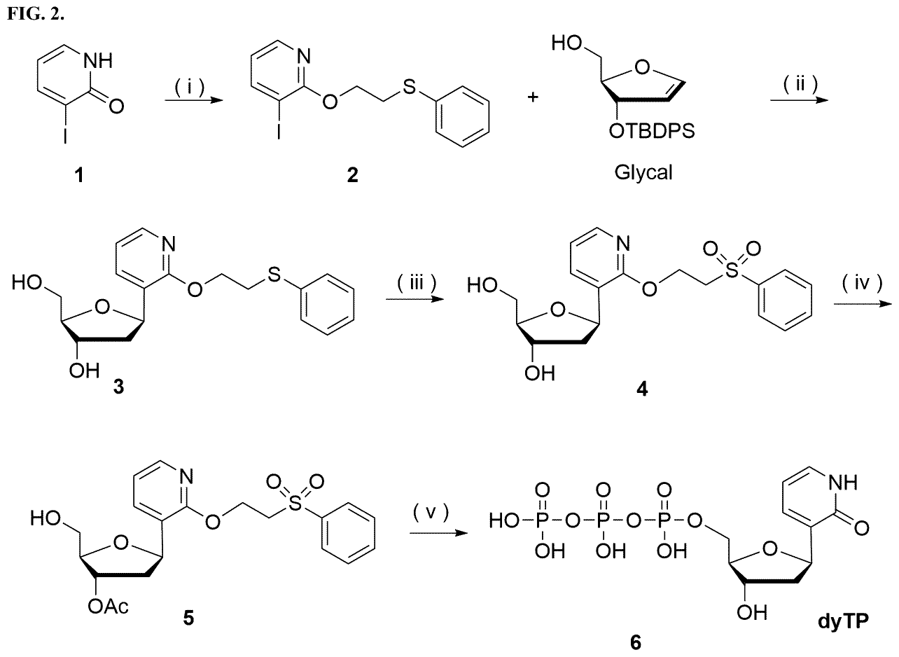

Example 1. Synthesis of dyTP. Solomon and Hopkins⁴⁵ delivered a stereo-controlled synthesis of dyTP from the acetonide of D-glyceraldehyde in a process that included a two-carbon homologation with diallyl zinc followed by reaction with a lithiated fluoropyridine. To implement BNA-Seq in a democratizable form, we required a simpler route. This started with a glycal derived from thymidine (FIG. 2). Palladium-catalyzed coupling of the glycal with an O-protected iodopyridinol gave the protected nucleoside analog. This was deprotected by oxidation of the sulfide followed by the formation of a triphosphate and base-catalyzed deprotection. The analytical data (NMR, FIRMS spectra) are summarized in supporting information.

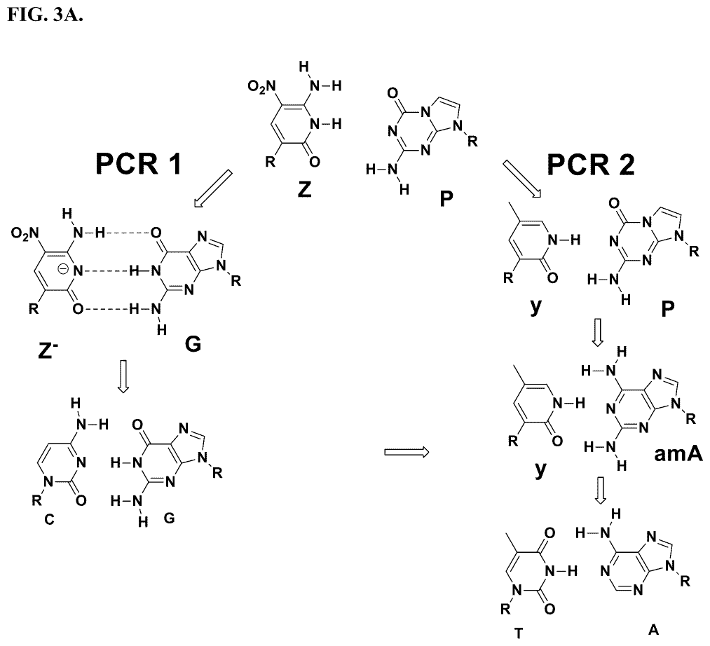

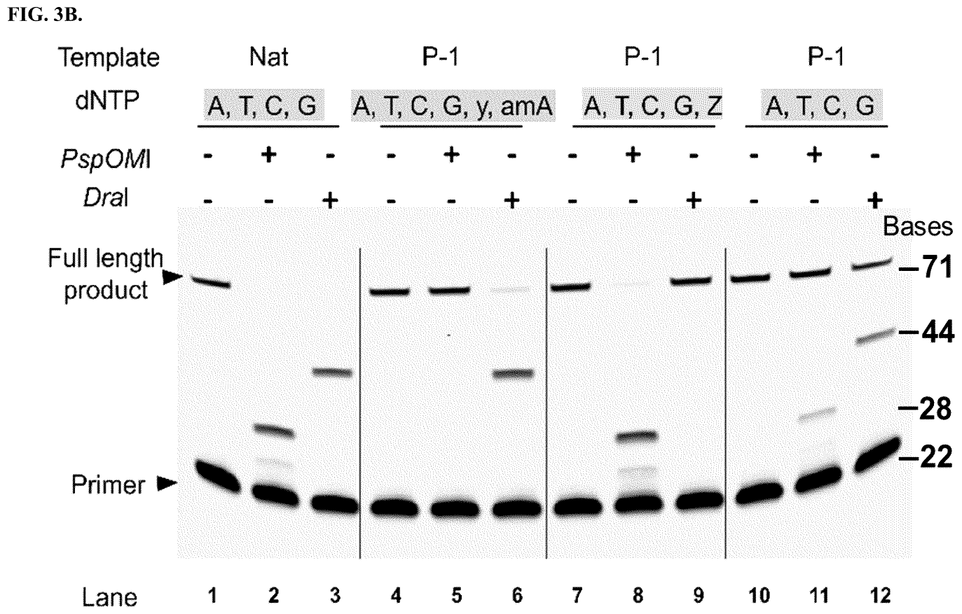

Example 2. Enzymology. In BNA-Seq, the biversal nucleotide dyTP serves as a substrate in the template-directed polymerization of a DNA template to match P. Here, the y:P pair is joined by two hydrogen bonds with a Watson-Crick geometry (FIG. 3A). 2-Aminoadenine (amA) triphosphate then operates in the next copying step to complete the transliteration. Amino-A pairs with y also via two hydrogen bonds with a Watson-Crick geometry and, in the next cycle, with T via three hydrogen bonds. Thus, in one amplification, Z:P pairs are transliterated to T:A pairs. To implement this application of the biversal concept, DNA molecules in Table 1 were made by solid phase synthesis and used with primers in PCR amplifications. The “Nat” sequence includes two restriction sites cut by PspOMI (GGGCCC) and DraI (TTTAAA). In contrast, AEGIS sequence “P-1” is designed to contain P within the restriction enzyme recognition sequence (GGPCCC, TTTPAA). Restriction enzymes do not cut these sites. Thus, if P is transliterated into either G or A during PCR, the PspOMI or (respectively) DraI sites are formed. This desired transliteration is then monitored by restriction digestion.

The Nat DNA template was amplified by PCR with the four standard triphosphates d(A, T, C, G)TP. In parallel, the P-1 DNA template was PCR amplified under three different conditions.

PCR Condition (1): dATP, dTTP, dCTP, dGTP and dZTP, to force P to be transliterated to G via a deprotonated Z:G match with Watson-Crick geometry.

PCR Condition (2): dATP, dTTP, dCTP, dGTP, dyTP and damATP, to force P to be transliterated to A via y:P and y:amA matches, both again with Watson-Crick geometry.

Regular PCR with four standard triphosphates, dATP, dTTP, dCTP and dGTP. Here. P is transliterated to G/A mixture, via P:C and P:T mismatch with the non-canonical and less robust.

Following initial amplification, products were diluted 2000-fold and then subjected to another PCR under standard PCR conditions. Subsequently, the PCR products were subjected to digestion by restriction endonucleases. These digested products were then analyzed by denaturing urea polyacrylamide gel electrophoresis.

In the first group, PCR amplicons derived from the Nat template in combination with a standard of 4-triphosphate PCR were digested by PspOMI and DraI. This resulted in a distinct, short band, observable on the urea-PAGE gel (Lanes 2 and 3, FIG. 3B). These data serve as a positive control, proving that restriction enzymes strategy could be used to analyze the products.

In the second group, where AEGIS template (P-1) was used under conditions where P might be transliterated to A, reducing the concentrations of dCTP and dGTP (to 0.1 mM) and maintaining the concentrations of dTTP and dATP (0.2 mM) do not significant increase the transliteration of P to A. However, when yTP (0.1 mM) and amATP (0.1 mM) were added into the PCR mixture, P was efficiently transliterated to A, as quantified by NGS, 86.6% (P-28) and 89.9% (P-44) (Table 2). Again, this shows that P first pairs with y, y then pairs with amA, amA then pair with T, and T then pair with A.

The concentration of dyTP in the PCR process was then systematically varied over a range of concentrations (0.1 mM to 0.5 mM). When the dyTP concentration was 0.1 mM, 86.6% of the initial P was transliterated to A. With increasing dyTP, the amount of P transliterating to A increased. Concentrations of dyTP above 0.3 mM did not increase the transliterated yield to over 96%. However, increased concentrations of damATP above 0.2 mM reduced the efficacy of the PCR.

Optimizing PCR with Condition 2 gave dATP (0.2 mM), dTTP (0.2 mM), dCTP (0.1 mM), dGTP (0.1 mM), dyTP (0.3 mM), and damATP (0.1 mM). Here, P-28 transliterated to A (94.7%) and P-44 transliterated to A (96.3%) were acceptable. The gel-based restriction enzyme strategy also shows these high levels of transliteration. Template (P-1) under optimized PCR condition 2 with six triphosphates d(A, T, C, G, y, amA)TP, resisted digestion by PspOMI (FIG. 3B, Lane 5), but was successfully digested by DraI (FIG. 3B, Lane 6). The amplicon was also sequenced by Sanger sequencing.

Analogous success was achieved when amplifying AEGIS template (P-1) under PCR Condition 1 (third group) with five triphosphates d(A, T, C, G, Z)TP. The products were digested by PspOMI (GGGCCC) (FIG. 3B, Lane 8), but not by DraI (TTTAAA), (FIG. 3B, Lane 9). These results show that template P nucleotide was transliterated to G under Condition 1, as expected from previous reports⁴³. Sanger sequencing was confirmed the sequence of the amplicons and NGS quantitated the ratio of transliteration products. Here, P-28 was transliterated to G (97.7%) and P-44 was transliterated to G (99.3%) (Table 2).

In the fourth set of experiments, the P-1 template was used under standard PCR conditions with the four standard triphosphates: d(A, T, C, G)TP. The amplicons were only partly digested by PspOMI and DraI (Lane 11 and Lane 12, FIG. 3B). This outcome suggests that the P nucleotide in a template underwent transliteration to an A/G mixture with standard PCR. The was confirmed by Sanger sequencing; NGS quantitated the transliteration ratios. P at position 28 was transliterated to A (57.3%) and G (42%), P at position 44 was transliterated to A (59.1%) and G (40.2%) (Table 2).

We then examined the fate of Z in the template. Here, no standard nucleotide forms a match with Watson-Crick geometry. Thus, dPTP was added at different concentrations (0.1 mM to 0.5 mM) to allow the first copy cycle to pair dPTP with template Z. A second template with two consecutive Z's showed that a 0.5 mM (dPTP) concentration gave the best transliteration (˜85%). Sanger sequencing and restriction enzyme digestion quantitated the results

Example 3. Sequencing AEGIS DNA contain both Z and P. A full sequencing workflow was then developed to sequence AEGIS DNA molecules that contain both Z and P. ZP-1 and ZP-2 templates under PCR Condition 1 transliterate their Z:P to C:G via pairing always with a Watson-Crick geometry. The same is true for PCR Condition 2, which transliterates Z:P to A:T via steps all involving pairs with Watson-Crick geometry. The resulting transliterated sequences were separately analyzing by Sanger sequencing. Bioinformatics reliably back-inferred the original sequences. NGS also quantified the results and were demonstrated using sequence logos. The transliteration ratio of all the base (excluded the primer region) in the templates of P-1, ZP-1, and ZP-2 after PCR 1 or PCR 2 are summarized in Table w. Standard bases (A, T, C, G) were called with >99% accuracy, showing that added non-standard triphosphates had no impact on those calls.

Example 4. Sequence context. We then asked whether the sequencing results obtained using the biversal nucleotide analog y were affected by neighbor sequences. Here, a library of sequences having all four standard nucleotides preceding and following an AEGIS Z or P (4096 combinations in all, —NNNZNNN— and —NNNPNNN—) were treated sequencing and NGS. No obvious context bias in sequence calls was seen. The detailed sequencing analytics is summarized in Wang et al. 2024 (op. cit).

Example 5. Applying BNA-Seq to a mixture of DNA molecules in a pool that arises from an AEGIS-LIVE evolution experiment. In AEGIS-LIVE, users preferably use BNA-Seq to deconvolute sequences from survivor pools with sequence diversity. To show the value of BNA-Seq in this application context, we prepared a mixture of DNA molecules with natural and Z and P nucleotides. This included five types of DNA molecules in a 1:1:1:1:1 ratio: Nat DNA (only ATCG nucleotides), ZZ DNA (ATCGZ), P-1 DNA (ATCGP), and ZP-1 and ZP-2 DNA (ATCGZP).

To test the sensitivity of this sequencing method, we prepared a dilution series from 1 pM to 0.001 pM and submitted each sample to BNA-seq. The results showed that all five templates were successfully read with high sensitivity even at 0.001 pM DNA concentration. Interestingly, the Nat, ZZ, and ZP-2 templates were sequenced at approximately 20%, corresponding to the initial preparation ratio. However, the P-1 template was read at a lower ratio of 7%, while the ZP-1 template was sequenced at a higher ratio of 30% compared to the initial 20%. This discrepancy may result from PCR efficiency bias, which is also common in general PCR.

Example 6. Steady-state kinetic assays. To quantitatively compare efficiencies of nucleotide incorporation in transfer PCR when DNA containing Z or P, steady-state kinetic assays were performed to characterize TaKaRa Taq polymerase (pseudo second-order rate constant), Vₘₐₓ/Kₘ (% min⁻¹·μM⁻¹) refer to the literature⁴⁷.

In this primer extension, single nucleotide incorporation (n+1 product formation) was followed with the final concentrations of dNTP ranging from 0.0013-100 μM. Kinetic parameters Kₘ and Vₘₐₓ were calculated. The insertion efficiencies of dCTP opposite dG is 478·min⁻¹·μM⁻¹. The insertion efficiencies of dZTP or dyTP opposite dPTP were 175 min⁻¹·μM⁻¹ and 1.26·min⁻¹·μM⁻¹ respectively. Moreover, no detectable incorporation of dTTP or dCTP opposite dP was observed under the same conditions. Insertion efficiencies of dPTP or dGTP opposite dZTP were 810·min⁻¹·μM⁻¹ and 152%·min⁻¹·μM⁻¹ respectively. Moreover, no detectable reactions was observed for dATP and damATP opposite to dZ under the same conditions.

TABLE 1
DNA sequences

| | | |
|---|---|---|
| Nat | SEQ ID | TAAGATGAGAGTTGAGGAGAGTTAAGGGCCCAACAGTCGATTTAAATA |
| | NO: 1 | TAGTAGTGTAAG TAGATAGTGGA |
| P-1 | SEQ ID | TAAGATGAGAGTTGAGGAGAGTTAAGGPCCCAACAGTCGATTTPAATAT |
| | NO: 2 | AGTAGTGTAAGT AGATAGTGGA |
| ZZ | SEQ ID | TAAGATGAGAGTTGAGGAGAGTTATCCAAGZTATAGGGCZZTTCAGTAT |
| | NO: 3 | AGTAGTGTAAGT AGATAGTGGA |
| ZP- | SEQ ID | TAAGATGAGAGTTGAGGAGAGTTACGTGZACGCPTPGTCAZCACAGTAT |
| 1 | NO: 4 | AGTAGTGTAAGT AGATAGTGGA |
| ZP- | SEQ ID | TAAGATGAGAGTTGAGGAGAGTTATCAPCGTAGCAZPCTTPTZATGTATA |
| 2 | NO: 5 | GTAGTGTAAGT AGATAGTGGA |
| Z- | SEQ ID | TAAGATGAGAGTTGAGGAGAGTTATNNNZNNNGTATAGTAGTGTAAGT |
| Ran | NO: 6 | AGATAGTGGA |
| P- | SEQ ID | TAAGATGAGAGTTGAGGAGAGTTATNNNPNNNGTATAGTAGTGTAAGT |
| Ran | NO: 7 | AGATAGTGGA |

TABLE 2
Transliteration results

| | | P-28 | P-44 | | P-28 | P-44 |
|---|---|---|---|---|---|---|
| PCR 1 | P to A | 2.2% | 0.4% | P to G | 97.7% | 99.3% |
| PCR 2 | P to A | 94.4% | 96.0% | P to G | 5.4% | 2.6% |
| Regular PCR | P to A | 57.3% | 59.1% | P to G | 42.0% | 40.2% |

TABLE 3
Transliteration results

| | PCR 1 | | PCR 2 |
|---|---|---|---|
| A to A | 99.6 ± 0.35 | A to A | 99.6 ± 0.2 |
| T to T | 99.6 ± 0.8 | T to T | 99.6 ± 0.8 |
| C to C | 99.7 ± 0.4 | C to C | 99.4 ± 0.3 |
| G to G | 99.7 ± 0.3 | G to G | 99.4 ± 0.5 |
| Z to C | 99.6 ± 0.2 | Z to T | 85.6 ± 4.6 |
| P to G | 97.0 ± 2.2 | P to A | 92.6 ± 2.6 |

### REFERENCES

(1) Hansch, C.; Fujita, T. p-σ-π Analysis. A Method for the Correlation of Biological Activity and Chemical Structure. J. Am. Chem. Soc. 1964, 86 (8), 1616-1626.
(2) Venkatraman, V.; Colligan, T. H.; Lesica, G. T.; Olson, D. R.; Gaiser, J.; Copeland, C. J.; Wheeler, T. J.; Roy, A. Drugsniffer: an open source workflow for virtually screening billions of molecules for binding affinity to protein targets. Front. Pharmacol. 2022, 13, 874746.
(3) Wildey, M. J.; Haunso, A.; Tudor, M.; Webb, M.; Connick, J. H. High-throughput screening. Annu. Rep. Med. Chem. 2017, 50, 149-195.
(4) Winter, G.; Griffiths, A. D.; Hawkins, R. E.; Hoogenboom, H. R. Making antibodies by phage display technology. Annu. Rev. Immunol. 1994, 12 (1), 433-455.
(5) Stumpp, M. T.; Dawson, K. M.; Binz, H. K. Beyond antibodies: the DARPin® drug platform. Biodrugs 2020, 34 (4), 423-433.
(6) (a) Baker, M. Blame it on the antibodies. Nature 2015, 521 (7552), 274. (b) Begley, C. G.; Ellis, L. M. Raise standards for preclinical cancer research. Nature 2012, 483 (7391), 531-533.
(7) Hayhurst, A.; Harris, W. J. Escherichia coli skp chaperone coexpression improves solubility and phage display of single-chain antibody fragments. Protein Expr. Purif 1999, 15 (3), 336-343.
(8) Acharya, V. V.; Chaudhuri, P. Modalities of protein denaturation and nature of denaturants. Int. J. Pharm. Sci. Res. 2021, 69 (2), 19-24.
(9) Gilbert, W. Origin of life: The RNA world. Nature 1986, 319 (6055), 618-618.
(10) Benner, S. A.; Ellington, A. D.; Tauer, A. Modern metabolism as a palimpsest of the RNA world. Proc. Natl. Acad. Sci. U.S.A. 1989, 86 (18), 7054-7058.
(11) White, H. B. Coenzymes as fossils of an earlier metabolic state. J. Mol. Evol. 1976, 7, 101-104.
(12) Ban, N.; Nissen, P.; Hansen, J.; Moore, P. B.; Steitz, T. A. The complete atomic structure of the large ribosomal subunit at 2.4 Å resolution. Science 2000, 289 (5481), 905-920.
(13) Altman, S. Enzymatic cleavage of RNA by RNA (Nobel lecture). Angew. Chem., Int. Ed. 1990, 29 (7), 749-758.
(14) Rich, A. On the problems of evolution and biochemical information transfer. In: Horizons in Biochemistry 1962, 103-126.
(15) Tuerk, C.; Gold, L. Systematic evolution of ligands by exponential enrichment: RNA ligands to bacteriophage T4 DNA polymerase. Science 1990, 249 (4968), 505-510.
(16) Lorsch, J. R.; Szostak, J. W. Chance and necessity in the selection of nucleic acid catalysts. Acc. Chem. Res. 1996, 29 (2), 103-110.
(17) Joyce, G. F. Directed evolution of nucleic acid enzymes. Annu. Rev. Biochem. 2004, 73 (1), 791-836.
(18) (a) Wang, B.; Pan, X.; Teng, I.-T.; Li, X.; Kobeissy, F.; Wu, Z.-Y.; Zhu, J.; Cai, G.; Yan, H.; Yan, X.; et al. Functional Selection of Tau Oligomerization-Inhibiting Aptamers. Angew. Chem., Int. Ed. 2024, 63 (18), e202402007. (b) Li, N.; Ebright, J. N.; Stovall, G. M.; Chen, X.; Nguyen, H. H.; Singh, A.; Syrett, A.; Ellington, A. D. Technical and biological issues relevant to cell typing with aptamers. J. Proteome Res. 2009, 8 (5), 2438-2448. (c) Byun, J. Recent progress and opportunities for nucleic acid aptamers. Life 2021, 11 (3), 193. (d) Rozenblum, G. T.; Lopez, V. G.; Vitullo, A. D.; Radrizzani, M. Aptamers: current challenges and future prospects. Expert Opin. Drug Discov. 2016, 11 (2), 127-135. (e) Wang, B.; Kobeissy, F.; Golpich, M.; Cai, G.; Li, X.; Abedi, R.; Haskins, W.; Tan, W.; Benner, S. A.; Wang, K. K. Aptamer Technologies in Neuroscience, Neuro-Diagnostics and Neuro-Medicine Development. Molecules 2024, 29 (5), 1124.
(19) Carrigan, M. A.; Ricardo, A.; Ang, D. N.; Benner, S. A. Quantitative analysis of a RNA-cleaving DNA catalyst obtained via in vitro selection. Biochemistry 2004, 43 (36), 11446-11459.
(20) Reader, J. S.; Joyce, G. F. A ribozyme composed of only two different nucleotides. Nature 2002, 420 (6917), 841-844.
(21) Hollenstein, M. DNA catalysis: the chemical repertoire of DNAzymes. Molecules 2015, 20 (11), 20777-20804.
(22) Wang, Y.; Ng, N.; Liu, E.; Lam, C. H.; Perrin, D. M. Systematic study of constraints imposed by modified nucleoside triphosphates with protein-like side chains for use in in vitro selection. Org. Biomol. Chem. 2017, 15 (3), 610-618.
(23) Wolk, S. K.; Mayfield, W. S.; Gelinas, A. D.; Astling, D.; Guillot, J.; Brody, E. N.; Janjic, N.; Gold, L. Modified nucleotides may have enhanced early RNA catalysis. Proc. Natl. Acad. Sci. U.S.A. 2020, 117 (15), 8236-8242.
(24) (a) Kimoto, M.; Nakamura, M.; Hirao, I. Post-ExSELEX stabilization of an unnatural-base DNA aptamer targeting VEGF165 toward pharmaceutical applications. Nucleic Acids Res. 2016, 44 (15), 7487-7494. (b) Kimoto, M.; Yamashige, R.; Matsunaga, K.-i.; Yokoyama, S.; Hirao, I. Generation of high-affinity DNA aptamers using an expanded genetic alphabet. Nat. Biotechnol. 2013, 31 (5), 453-457.
(25) Gold, L.; Ayers, D.; Bertino, J.; Bock, C.; Bock, A.; Brody, E. N.; Carter, J.; Dalby, A. B.; Eaton, B. E.; Fitzwater, T.; et al. Aptamer-Based Multiplexed Proteomic Technology for Biomarker Discovery. PLoS One 2010, 5 (12), e15004.
(26) Benner, S.; Allemann, R. K.; Ellington, A.; Ge, L.; Glasfeld, A.; Leanz, G.; Krauch, T.; MacPherson, L.; Moroney, S.; Piccirilli, J. Natural selection, protein engineering, and the last riboorganism: rational model building in biochemistry. Cold Spring Harbor Symp. Quant. Biol. 1987, 52, 53-63.
(27) Hoshika, S.; Leal, N. A.; Kim, M.-J.; Kim, M.-S.; Karalkar, N. B.; Kim, H.-J.; Bates, A. M.; Watkins Jr, N. E.; SantaLucia, H. A.; Meyer, A. J. Hachimoji DNA and RNA: A genetic system with eight building blocks. Science 2019, 363 (6429), 884-887.
(28) Benner, S. A. Rethinking nucleic acids from their origins to their applications. Philos. Trans. R. Soc. B. 2023, 378 (1871), 20220027.
(29) Benner, S. A.; Kim, H.-J.; Biondi, E. Prebiotic chemistry that could not not have happened. Life 2019, 9 (4), 84.
(30) (a) Yang, Z.; Chen, F.; Chamberlin, S. G.; Benner, S. A. Expanded genetic alphabets in the polymerase chain reaction. Angew. Chem., Int. Ed. 2010, 49 (1), 177. (b) Hoshika, S.; Chen, F.; Leal, N. A.; Benner, S. A. Artificial Genetic Systems: Self-Avoiding DNA in PCR and Multiplexed PCR. Angew. Chem. Int. Ed. 2010, 49 (32), 5554-5557.
(31) Jerome, C. A.; Hoshika, S.; Bradley, K. M.; Benner, S. A.; Biondi, E. In vitro evolution of ribonucleases from expanded genetic alphabets. Proc. Natl. Acad. Sci. U.S. Pat. No. 2,022,119 (44), e2208261119.
(32) Zhang, L.; Yang, Z.; Sefah, K.; Bradley, K. M.; Hoshika, S.; Kim, M.-J.; Kim, H.-J.; Zhu, G.; Jiménez, E.; Cansiz, S.; et al. Evolution of Functional Six-Nucleotide DNA. J. Am. Chem. Soc. 2015, 137 (21), 6734-6737.
(33) Biondi, E.; Lane, J. D.; Das, D.; Dasgupta, S.; Piccirilli, J. A.; Hoshika, S.; Bradley, K. M.; Krantz, B. A.; Benner, S. A. Laboratory evolution of artificially expanded DNA gives redesignable aptamers that target the toxic form of anthrax protective antigen. Nucleic Acids Res. 2016, 44 (20), 9565-9577.
(34) (a) Chaput, J. C.; Switzer, C. A DNA pentaplex incorporating nucleobase quintets. Proc. Natl. Acad. Sci. U.S.A. 1999, 96 (19), 10614-10619. (b) Hoshika, S.; Singh, I.; Switzer, C.; Molt Jr, R. W.; Leal, N. A.; Kim, M.-J.; Kim, M.-S.; Kim, H.-J.; Georgiadis, M. M.; Benner, S. A. “Skinny” and “Fat” DNA: two new double helices. J. Am. Chem. Soc. 2018, 140 (37), 11655-11660.
(35) Wang, B.; Rocca, J. R.; Hoshika, S.; Chen, C.; Yang, Z.; Esmaeeli, R.; Wang, J.; Pan, X.; Lu, J.; Wang, K. K.; Cao, Y. C.; Tan, W.; Benner, S. A. A folding motif formed with an expanded genetic alphabet. Nat. Chem. 2024, 16 (10), 1715-1722.
(36) Kim, H.-J.; Chen, F.; Benner, S. A. Synthesis and properties of 5-cyano-substituted nucleoside analog with a donor-donor-acceptor hydrogen-bonding pattern. J. Org. Chem. 2012, 77 (7), 3664-3669.
(37) Hendrickson, C. L.; Devine, K. G.; Benner, S. A. Probing minor groove recognition contacts by DNA polymerases and reverse transcriptases using 3-deaza-2′-deoxyadenosine. Nucleic Acids Res. 2004, 32 (7), 2241-2250.
(38) Chen, F.; Gaucher, E. A.; Leal, N. A.; Hutter, D.; Havemann, S. A.; Govindarajan, S.; Ortlund, E. A.; Benner, S. A. Reconstructed evolutionary adaptive paths give polymerases accepting reversible terminators for sequencing and SNP detection. Proc. Natl. Acad. Sci. U.S.A. 2010, 107 (5), 1948-1953.
(39) (a) Laos, R.; Shaw, R.; Leal, N. A.; Gaucher, E.; Benner, S. Directed evolution of polymerases to accept nucleotides with nonstandard hydrogen bond patterns. Biochemistry 2013, 52 (31), 5288-5294. (b) Laos, R.; Thomson, J. M.; Benner, S. A. DNA polymerases engineered by directed evolution to incorporate non-standard nucleotides. Front. Microbiol. 2014, 5, 105696.
(40) (a) Lutz, M. J.; Horlacher, J.; Benner, S. A. Recognition of 2′-deoxyisoguanosine triphosphate by HIV-1 reverse transcriptase and mammalian cellular DNA polymerases. Bioorg. Med. Chem. Lett. 1998, 8 (5), 499-504. (b) Sismour, A. M.; Lutz, S.; Park, J. H.; Lutz, M. J.; Boyer, P. L.; Hughes, S. H.; Benner, S. A. PCR amplification of DNA containing non-standard base pairs by variants of reverse transcriptase from Human Immunodeficiency Virus-1. Nucleic Acids Res. 2004, 32 (2), 728-735. (c) Leal, N. A.; Kim, H.-J.; Hoshika, S.; Kim, M.-J.; Carrigan, M. A.; Benner, S. A. Transcription, reverse transcription, and analysis of RNA containing artificial genetic components. ACS Synth. Biol. 2015, 4 (4), 407-413.
(41) (a) Wang, X.; Hoshika, S.; Peterson, R. J.; Kim, M.-J.; Benner, S. A.; Kahn, J. D. Biophysics of artificially expanded genetic information systems. Thermodynamics of DNA duplexes containing matches and mismatches involving 2-amino-3-nitropyridin-6-one (Z) and imidazo [1, 2-a]-1,3,5-triazin-4 (8H) one (P). ACS Synth. Biol. 2017, 6 (5), 782-792. (b) Pham, T. M.; Miffin, T.; Sun, H.; Sharp, K. K.; Wang, X.; Zhu, M.; Hoshika, S.; Peterson, R. J.; Benner, S. A.; Kahn, J. D.; et al. DNA Structure Design Is Improved Using an Artificially Expanded Alphabet of Base Pairs Including Loop and Mismatch Thermodynamic Parameters. ACS Synth. Biol. 2023, 12 (9), 2750-2763.
(42) Yang, Z.; Chen, F.; Alvarado, J. B.; Benner, S. A. Amplification, mutation, and sequencing of a six-letter synthetic genetic system. J. Am. Chem. Soc. 2011, 133 (38), 15105-15112.
(43) Wang, B.; Bradley, K. M.; Kim, M.-J.; Laos, R.; Chen, C.; Gerloff, D. L.; Manfio, L.; Yang, Z.; Benner, S. A. Enzyme-assisted high throughput sequencing of an expanded genetic alphabet at single base resolution. Nat. Commun. 2024, 15 (1), 4057.
(44) Yang, Z.; Kim, H.-J.; Le, J. T.; McLendon, C.; Bradley, K. M.; Kim, M.-S.; Hutter, D.; Hoshika, S.; Yaren, O.; Benner, S. A. Nucleoside analogs to manage sequence divergence in nucleic acid amplification and SNP detection. Nucleic Acids Res. 2018, 46 (12), 5902-5910.
(45) (a) Solomon, M. S.; Hopkins, P. B. Stereocontrolled syntheses of C-linked deoxyribosides of 2-hydroxypyridine and 2-hydroxyquinoline. Tetrahedron Lett. 1991, 32 (28), 3297-3300. (b) Ishikawa, M.; Hirao, I.; Yokoyama, S. Synthesis of 3-(2-deoxy-β-d-ribofuranosyl)pyridin-2-one and 2-amino-6-(N,N-dimethylamino)-9-(2-deoxy-β-d-ribofuranosyl)purine derivatives for an unnatural base pair. Tetrahedron Lett. 2000, 41 (20), 3931-3934.
(46) Ishikawa, M.; Hirao, I.; Yokoyama, S. Synthesis of 3-(2-deoxy-β-D-ribofuranosyl) pyridin-2-one and 2-amino-6-(N, N-dimethylamino)-9-(2-deoxy-β-D-ribofuranosyl) purine derivatives for an unnatural base pair. Tetrahedron Lett. 2000, 41 (20), 3931-3934.
(47) Geyer, C. R.; Battersby, T. R.; Benner, S. A. Nucleobase pairing in expanded Watson-Crick-like genetic information systems. Structure 2003, 11 (12), 1485-1498.
(48) Lipps, H. J.; Rhodes, D. G-quadruplex structures: in vivo evidence and function. Trends Cell Biol. 2009, 19 (8), 414-422.
(49) Roberts, C.; Chaput, J. C.; Switzer, C. Beyond guanine quartets: cation-induced formation of homogenous and chimeric DNA tetraplexes incorporating iso-guanine and guanine. Chem. Biol. 1997, 4 (12), 899-908.

What is claimed is:

1. A composition of matter that comprises two oligonucleotides, wherein said oligonucleotides are hybridized in a duplex, wherein the strands of said two oligonucleotides have an antiparallel orientation, wherein each nucleotide in one strand forms a hydrogen bonded pair with an oligonucleotide in the other strand, wherein at least one of the inventive pairs is a pair between two heterocycles, wherein said heterocycles are either 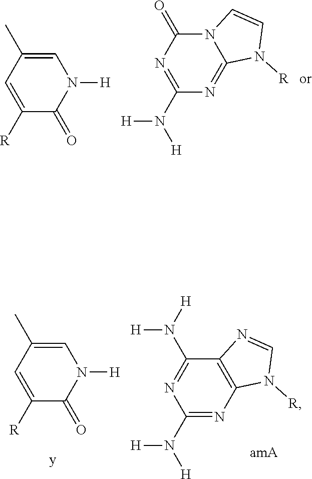
wherein R is the point of attachment of the heterocycles to the said oligonucleotides.

2. A process wherein an inventive pair in a DNA duplex is formed by polymerase-catalyzed template-directed incorporation of 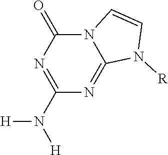
opposite 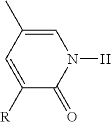
in a template, or incorporation of 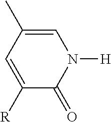
opposite 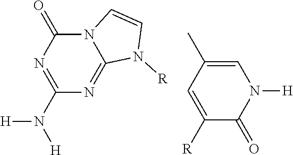
in a template, or incorporation of 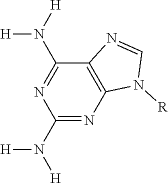
opposite 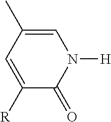
in a template, or incorporation of 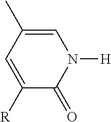
opposite 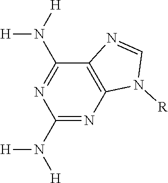
in a template, wherein said inventive pairs are either 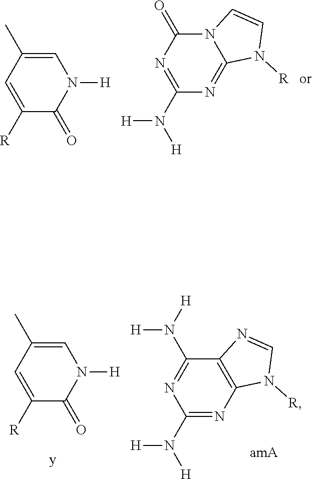
wherein R is the point of attachment of the heterocycles to the said oligonucleotides, and wherein said process comprises the step of incubating a template comprising one of the heterocycles in one of the inventive pairs with a nucleoside triphosphate comprising the second heterocycle in the inventive pair, together with a DNA or RNA polymerase, or a reverse transcriptase.

3. The process of claim 2, wherein said polymerase is a DNA polymerase.

4. A method for sequencing a nucleic acid containing standard and non-standard nucleotides, comprising: (a) providing a nucleic acid template containing one or more non-standard nucleotides selected from an artificially expanded genetic alphabet (AEGIS), (b) amplifying the nucleic acid using polymerase chain reaction (PCR) with triphosphates comprising standard nucleotides and a biversal nucleotide analog, wherein the biversal nucleotide forms Watson-Crick pairs with at least one standard nucleotide and one non-standard nucleotide, (c) transliterating the non-standard nucleotides into standard nucleotide sequences during the amplification process, (d) sequencing the amplified product using high-throughput sequencing technologies, and inferring the original nucleic acid sequence by bioinformatics analysis of transliterated sequences.

5. The method of claim 4, wherein the biversal nucleotide analog is a pyridine-2-one that forms an inventive pair with both 2-amino-2′-deoxyadenosine and 2-amino-8-imidazo-[1,2a]-1,3,5-triazin-[8H]-4-one.


SEQUENCE LISTING (7 sequences)

SEQ ID NO: 1 - DNA, 71 bp, unidentified
  source 1..71: mol_type = unassigned DNA; note = synthetic construct
taagatgagagttgaggagagttaagggcccaacagtcgatttaaatatagtagtgtaagtagatagtgga

SEQ ID NO: 2 - DNA, 71 bp, unidentified
  source 1..71: mol_type = unassigned DNA; note = synthetic construct
  modified_base 28: mod_base = OTHER; note = 2-amino-8-imidazo-[1,2a]-1,3,5-triazin-[8H]-4-one
  modified_base 44: mod_base = OTHER; note = 2-amino-8-imidazo-[1,2a]-1,3,5-triazin-[8H]-4-one
taagatgagagttgaggagagttaaggncccaacagtcgatttnaatatagtagtgtaagtagatagtgga

SEQ ID NO: 3 - DNA, 71 bp, unidentified
  source 1..71: mol_type = unassigned DNA; note = synthetic construct
  modified_base 31: mod_base = OTHER; note = 6-amino-5-nitro-[1H]-pyridin-2-one
  modified_base 40: mod_base = OTHER; note = 6-amino-5-nitro-[1H]-pyridin-2-one
  modified_base 41: mod_base = OTHER; note = 6-amino-5-nitro-[1H]-pyridin-2-one
taagatgagagttgaggagagttatccaagntatagggcnnttcagtatagtagtgtaagtagatagtgga

SEQ ID NO: 4 - DNA, 71 bp, unidentified
  source 1..71: mol_type = unassigned DNA; note = synthetic construct
  modified_base 29: mod_base = OTHER; note = 6-amino-5-nitro-[1H]-pyridin-2-one
  modified_base 41: mod_base = OTHER; note = 6-amino-5-nitro-[1H]-pyridin-2-one
  modified_base 34: mod_base = OTHER; note = 2-amino-8-imidazo-[1,2a]-1,3,5-triazin-[8H]-4-one
  modified_base 36: mod_base = OTHER; note = 2-amino-8-imidazo-[1,2a]-1,3,5-triazin-[8H]-4-one
taagatgagagttgaggagagttacgtgnacgcntngtcancacagtatagtagtgtaagtagatagtgga

SEQ ID NO: 5 - DNA, 71 bp, unidentified
  source 1..71: mol_type = unassigned DNA; note = synthetic construct
  modified_base 28: mod_base = OTHER; note = 2-amino-8-imidazo-[1,2a]-1,3,5-triazin-[8H]-4-one
  modified_base 37: mod_base = OTHER; note = 2-amino-8-imidazo-[1,2a]-1,3,5-triazin-[8H]-4-one
  modified_base 41: mod_base = OTHER; note = 2-amino-8-imidazo-[1,2a]-1,3,5-triazin-[8H]-4-one
  modified_base 36: mod_base = OTHER; note = 6-amino-5-nitro-[1H]-pyridin-2-one
  modified_base 43: mod_base = OTHER; note = 6-amino-5-nitro-[1H]-pyridin-2-one
taagatgagagttgaggagagttatcancgtagcanncttntnatgtatagtagtgtaagtagatagtgga

SEQ ID NO: 6 - DNA, 58 bp, unidentified
  source 1..58: mol_type = unassigned DNA; note = synthetic construct
  modified_base 29: mod_base = OTHER; note = 6-amino-5-nitro-[1H]-pyridin-2-one
taagatgagagttgaggagagttatnnnnnnngtatagtagtgtaagtagatagtgga

SEQ ID NO: 7 - DNA, 58 bp, unidentified; identical to SEQ ID NO: 6
  source 1..58: mol_type = unassigned DNA; note = synthetic construct
  modified_base 29: mod_base = OTHER; note = 2-amino-8-imidazo-[1,2a]-1,3,5-triazin-[8H]-4-one